(19) United States
(12) Patent Application Publication
(10) Pub. No.: US 2025/0268773 A1
(43) Pub. Date: Aug. 28, 2025

(54) DEVICE FOR CORRECTING POSTURE

(71) Applicant: Bun Hee CHOI, Coquitlam (CA)

(72) Inventor: Bun Hee CHOI, Coquitlam (CA)

(21) Appl. No.: 18/637,950

(22) Filed: Apr. 17, 2024

(30) Foreign Application Priority Data
Feb. 27, 2024 (KR) 10-2024-0028007

(51) Int. Cl. A61G 13/12; A61G 13/00

(52) U.S. Cl. CPC A61G 13/121; A61G 13/009; A61G 13/1285; A61G 2200/325

(57) ABSTRACT

A device for correcting posture is proposed. Specifically, to relieve body imbalances such as turtle neck syndrome and round shoulder without damaging the body through stretching using gravity, the device for correcting posture includes a face support module supporting the face of a user lying prone so that the user's head is located higher than the feet. The face support module includes a face support cushion having a cross section shaped into a horseshoe to support the edge of the facial area and jaws of the user, and a link member configured to tilt the face support cushion so that the face support cushion is inclined downward in a forward direction where the top of the user's head faces. The user lying prone can be maintained in a posture in which the jaws are pulled and the ears and shoulders are located in the same level.

### CROSS REFERENCE TO RELATED APPLICATION

The present application claims priority to Korean Patent Application No. 10-2024-0028007, filed Feb. 27, 2024, the entire contents of which is incorporated herein for all purposes by this reference.

### BACKGROUND

### 1. Technical Field

The present disclosure relates to a device for correcting posture and, more particularly, to a device for correcting posture which is capable of reducing physical imbalance such as specifically turtle neck syndrome and round shoulder and improving pain relief and joint mobility.

### 2. Description of the Related Art

Modern people frequently use smartphones or computers, and at time of use, a user lowers the head and has a posture with the neck stretched forward. This posture causes shoulder impingement syndrome or round shoulder, which cause degenerative diseases such as rotator cuff tear, as well as turtle neck syndrome, which causes pain due to stretching of the muscles and the ligaments of the neck and the shoulders.

Turtle neck syndrome and round shoulder cause the user to suffer knotted shoulder and back muscles, headaches, and numbness in arms. Moreover, turtle neck syndrome and round shoulder reduce work and learning efficiency, make the user feel tired easily, and in rare cases, cause insomnia or dizziness. Furthermore, the user with turtle neck syndrome and round shoulder is more likely to suffer from scoliosis and pelvic distortion, thus causing imbalance throughout the body.

To correct turtle neck syndrome and round shoulder, neck retractors, neck stretchers, and round back correction bands are used. However, since the neck retractor straightens only the neck part of the unbalanced whole body, the neck retractor causes damage to the back and waist, and limits movement of the neck muscles that are stiff due to turtle neck syndrome, thus causing the muscles to have even more stiffness. Furthermore, although the neck stretcher induces a normal curve of the cervical spine, there is a risk of worsening turtle neck syndrome. The round back correction band supports the muscles around the shoulders and the chest, but it is difficult to support the shoulder joint, which causes pain.

### DOCUMENTS OF RELATED ART

### (Patent Document 1) Korean Utility Model Registration No. 20-0475488 ‘Pillow for Prone Posture’

### SUMMARY

The present disclosure has been made keeping in mind the above problems, and the present disclosure is intended to propose a device for correcting posture, the device being capable of inhibiting damage to the user's body with stretching using gravity and reducing body imbalances such as turtle neck syndrome and round shoulder.

In order to achieve the above objective, according to the present disclosure, there is provided a device for correcting posture, the device including: a face support module supporting the face of a user lying prone so that the user's head is located higher than the feet, wherein the face support module may include a face support cushion having a cross section shaped into a horseshoe to support the edge of the facial area and the jaws of the user, and a link member configured to tilt the face support cushion so that the face support cushion may be inclined downward in a forward direction where the top of the user's head faces, so that the user lying prone may be maintained in a posture in which the jaws may be pulled and the ears and shoulders may be located on the same level.

The face support module may include a base member placed on floor, and a face support fixing plate provided above the base member and fixed to the face support cushion at an upper surface thereof, and the link member may include first joint links and second joint links each having first ends respectively shaft-coupled to portions of the base member located below a rear end of the face support fixing plate, and the first joint links may be formed longer than the second joint links and second ends of the first joint links may be rotatably coupled to a lower surface of a front end of the face support fixing plate, and second ends of the second joint links may be rotatably coupled to a lower surface of a middle portion of the face support fixing plate.

The first joint links may have a form of a straight bar, the second joint links may have a form of a bent bar in which a first end portion may have a predetermined length and an angle between the first end portion and the remaining portion may be an obtuse angle, the first end portions of the second joint links may have moving guide holes each formed in a longitudinal direction thereof, and the shaft-coupling points between the second joint links and the base member may be movable along the moving guide holes.

The base member may include a base frame having a ‘ [symbol] ’ shape, a rotation shaft connecting opposite ends of the base frame, and penetrating through the first ends of the first joint links, which may be provided inside close to the opposite ends of the base frame, and through the moving guide holes of the second joint links provided inside the first joint links, a bushing provided between the second joint links that face each other while the rotation shaft penetrates in a longitudinal direction thereof, and a lever provided at one end of the rotation shaft protruding outward from one end of the base frame, which may allow the first joint links, the second joint links, and the bushing to come into close contact with each other or release them from the close contact state to shaft-rotate the first joint links and the second joint links.

The device for correcting posture may include: a shoulder support module supporting each shoulder of the user lying prone, wherein the shoulder support module may include a shoulder support cushion on which each shoulder may be placed, and a height adjusting stand adjusting height of the shoulder support cushion.

According to the present disclosure, the face support cushion may be tilted to be inclined downward in a forward direction by the link member, so that the user, who lies prone with the head located higher than the feet and the face supported by the face support module, can maintain the jaws pulled and the ears and shoulders located at the same level. During while maintaining this posture, since stretching using gravity is performed, body imbalances such as turtle neck syndrome and round shoulder are reduced without stretching that harms the body, so that pain relief and joint mobility can be improved.

Furthermore, the height of the shoulder support module can be adjusted so that the shoulders of the user, who lies prone with the head located higher than the feet and the face supported by the face support module, are in a straight posture, thus maximizing the effect of correcting turtle neck syndrome and round shoulder.

### BRIEF DESCRIPTION OF THE DRAWINGS

FIG. 1 is a perspective view showing a face support module of a device for correcting posture according to the present disclosure;

FIG. 2 is a plane view showing the face support module of the device for correcting posture according to the present disclosure;

FIG. 3 is a lower-perspective view showing the face support module of the device for correcting posture according to the present disclosure;

FIG. 4 is an exploded-perspective view showing the face support module of the device for correcting posture according to the present disclosure;

FIG. 5 is an exploded-side view showing the face support module of the device for correcting posture according to the present disclosure;

FIG. 6 is a perspective view showing a base frame applied to the face support module of the device for correcting posture according to the present disclosure;

FIG. 7 is a perspective view showing a first joint link applied to the face support module of the device for correcting posture according to the present disclosure;

FIGS. 8A and 8B are example views showing height and angle adjustment of the face support module of the device for correcting posture according to the present disclosure;

FIG. 9 is an example view showing a user lying prone using the device for correcting posture according to the present disclosure;

FIG. 10 is an example view showing a lever applied to the face support module of the device for correcting posture according to the present disclosure, which may be laterally turned;

FIG. 11 is a sectional view showing coupling relationship of components, when a first joint link and a second joint link are in a fixed state by operation of the lever applied to the face support module of the device for correcting posture according to the present disclosure;

FIG. 12 is a sectional view showing coupling relationship of the components when the first joint link and the second joint link are in a rotatable state by operation of the lever applied to the face support module of the device for correcting posture according to the present disclosure;

FIG. 13 is an example view showing the lever applied to the face support module of the device for correcting posture according to the present disclosure, which may be longitudinally turned; and

FIG. 14 is a front view showing a shoulder support module of the device for correcting posture according to the present disclosure.

### DETAILED DESCRIPTION

According to the present disclosure, to relieve body imbalances such as turtle neck syndrome and round shoulder without damaging the body through stretching using gravity, there is provided a device for correcting posture, which includes a face support module supporting the face of a user lying prone so that the user's head is located higher than the feet. The face support module includes a face support cushion having a cross section shaped into a horseshoe to support the edge of the facial area and jaws of the user, and a link member configured to tilt the face support cushion so that the face support cushion is inclined downward in a forward direction where the top of the user's head faces. The user lying prone can be maintained in a posture in which the jaws are pulled and the ears and shoulders are located in the same level.

The scope of the patent right of the present disclosure is not limited to the embodiments described below, and various modifications, additions and substitutions of the present disclosure are possible without departing from the scope and spirit of the present disclosure by one of ordinary skill in the art to which this invention belongs.

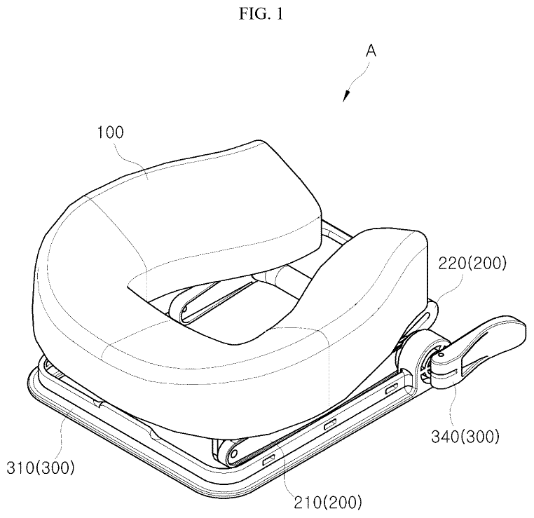

Hereinbelow, a device for correcting posture according to the present disclosure will be described in detail with reference to accompanying drawings of FIGS. 1 to 14.

The device for correcting posture according to the present disclosure includes a face support module A supporting the face of a user lying prone. At this point, the user lying prone is in a prone posture such that the user's head is located higher than the feet. In other words, the prone state means that the user lies prone to be inclined upward with respect to the floor surface toward the front side, i.e., in a direction where the top of the user's head lying prone faces. At this point, an angle between the user lying prone and the floor surface may range from 10 to 20°, but the angle is not limited to the range, and may vary according to the circumferences of the upper and lower body of the user. However, preferably, the user, who lies prone while being inclined upward in the forward direction with the face supported by the face support module A, may be supported at least the pelvis, shoulders, and face at the same time to further strengthen the balance of the pelvis and shoulders, and more preferably, may be supported at the shoulders so that both shoulders are straightened in order to maximize the effect of correcting turtle neck syndrome and round shoulder.

As described above, as shown in FIGS. 1 to 5, the face support module A supporting the face of the user lying prone includes a face support cushion 100 and a link member 200, and may include a base member 300 and a face support fixing plate 400.

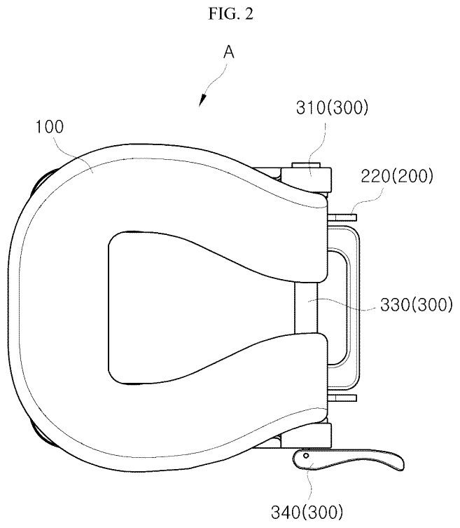

As shown in FIGS. 1 and 2, the face support cushion 100 has a cross section shaped into a horseshoe like a ‘C’ shape and an open portion thereof is disposed to face the rear side, thus supporting an edge of the facial area and the jaws of the user lying prone. Preferably, the face support cushion 100 has enough strength to support the face while having a vertically predetermined thickness. Furthermore, the face support cushion 100 is formed with the upper surface inclined downward in an inward direction from an outer edge thereof, so it is preferable for the face support cushion 100 to accommodate a part of the face and maintain the face in the stably placed state.

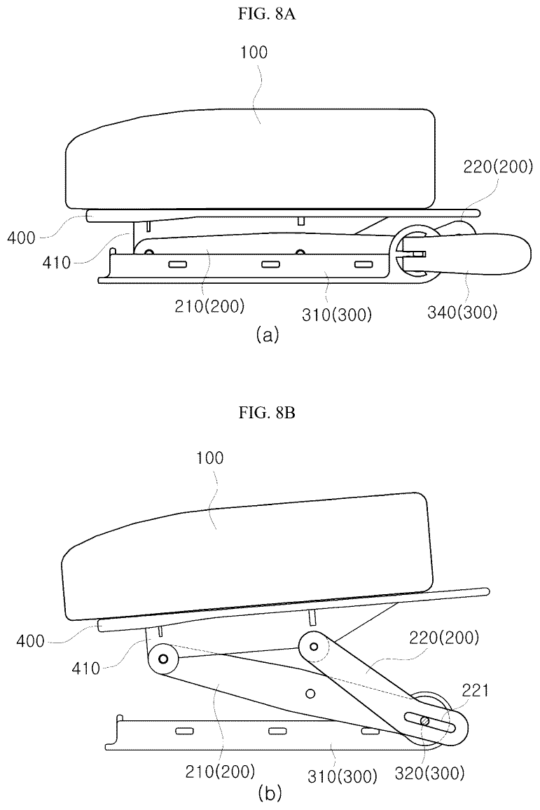

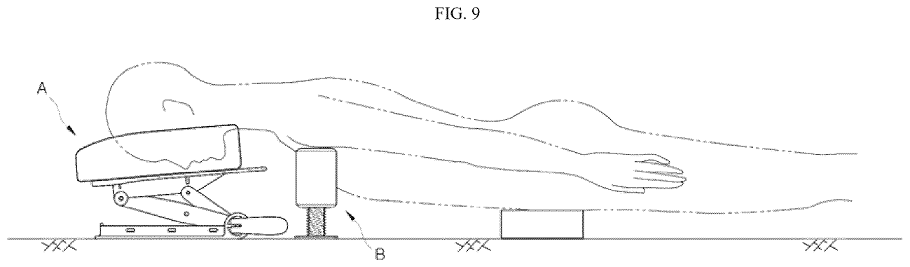

As shown in FIG. 8B, the link member 200 is configured to be inclined downward in a forward direction that is a direction where the top of the user's head with the face on the face support cushion 100 faces. In consideration of the user's body and the degree by which the correction is required, the link member 200 may be configured to allow the face support cushion 100 to be disposed horizontally or the face support cushion 100 to be disposed downward in the forward direction. In other words, as shown in FIG. 9, the link member 200 is provided to adjust the height and angle of the face support cushion 100 so that the jaws of the user lying prone are pulled and the ears and shoulders are located at the same level. The posture of the user lying prone is the same as a user rising with a normal neck posture without turtle neck syndrome, and has a different body angle therefrom.

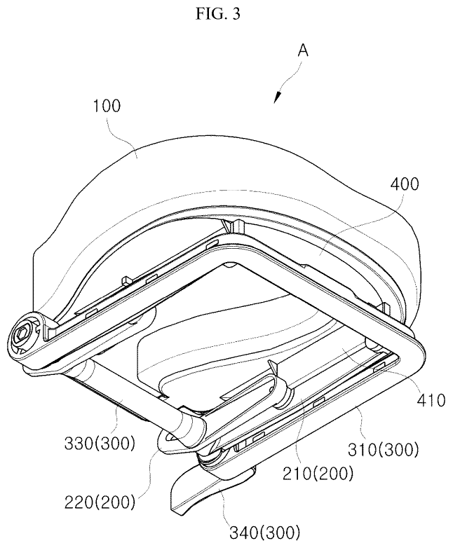

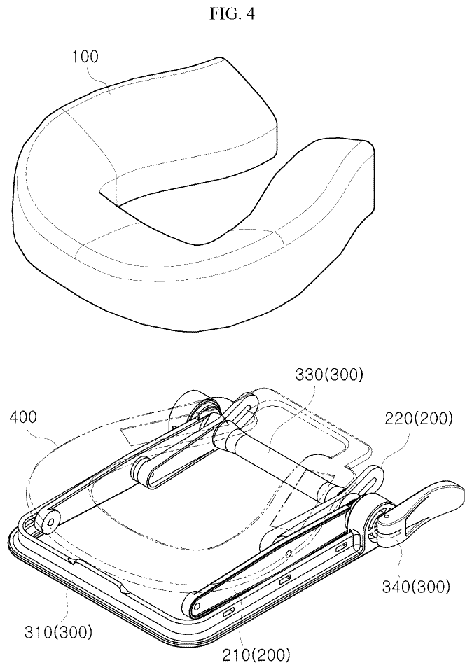

As described above, the link member 200, which can adjust the height and angle of the face support cushion 100, is connected to the base member 300 and the face support fixing plate 400 as shown in FIGS. 3 and 4.

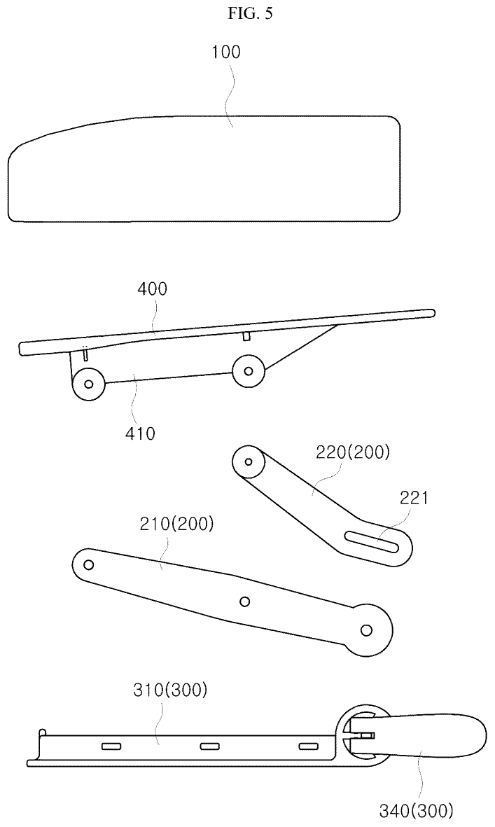

The base member 300 is placed on the floor and supports the face support module A, and the face support fixing plate 400 is placed above the base member 300 and may be fixed to the face support cushion 100, at an upper surface thereof. A detailed structure of the base member 300 will be described below, and the face support fixing plate 400 may be formed into a plate of a horseshoe shape from a plane view as an example, and the face support cushion 100 may be removably coupled to the upper surface of the face support fixing plate 400. Furthermore, as shown in FIGS. 3 and 5, the face support fixing plate 400 may have link coupling protrusions 410, which may be coupled to the link member 200, by protruding on opposite portions of a lower surface thereof. As an example, each of the link coupling protrusions 410 may have a form of a longitudinally long plate perpendicularly disposed on the lower surface of the face support fixing plate 400. In addition, the face support fixing plate 400 may have an adjustment handle connecting the opposite ends thereof located at the rear side and protruding outward, and the jaws of the users with the face on the face support cushion 100 may be easily adjusted to be pulled by the adjustment handle.

As shown in FIGS. 3 to 5, the link member 200 may include first joint links 210 and second joint links 220 with first ends respectively shaft-coupled to portions of the base member 300 located below a rear end of the face support fixing plate 400.

As an example, the first joint links 210 may have a form of a straight bar, and may be formed longer than the second joint links 220. The first joint links 210 have the first ends through which a rotation shaft 320 constituting the base member 300 may penetrate, and second ends which may be rotatably coupled to a lower surface of a front end of the face support fixing plate 400 (i.e., front ends of the link coupling protrusions 410). Furthermore, the first ends of the first joint links 210 may be fitted over the rotation shaft 320, and may be rotatably coupled to a first end or a second end of the base frame 310 constituting the base member 300.

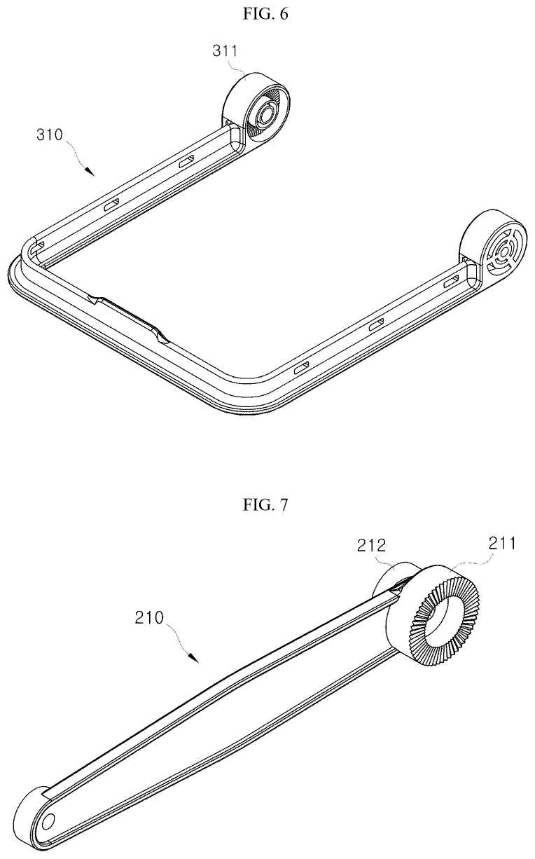

As an example, as shown in FIGS. 6 to 11, the base member 300 may include the base frame 310 having a form of ‘ [symbol] ’ and placed on the floor surface, and the rotation shaft 320 connecting one end to the other end of the base frame 310 and penetrating the first ends of the first joint links 210 and the first ends of the second joint links 220. At this point, the first ends of the first joint links 210 may be respectively located inside close to the opposite ends of the base frame 310, and the first ends of the second joint links 220 may be respectively located inside of the first joint links 210.

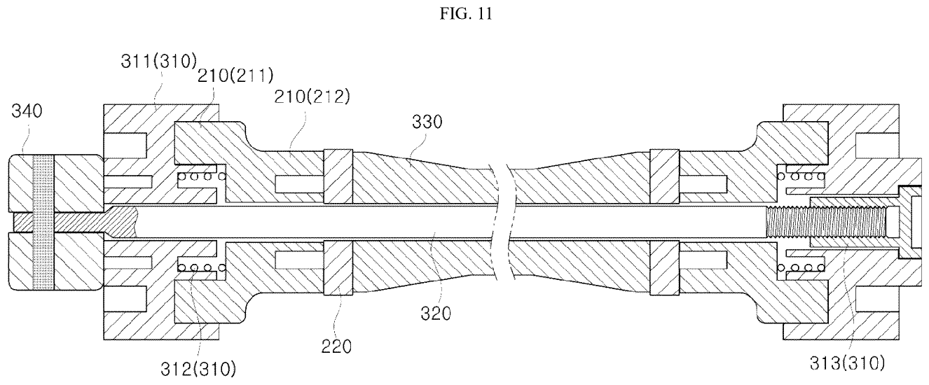

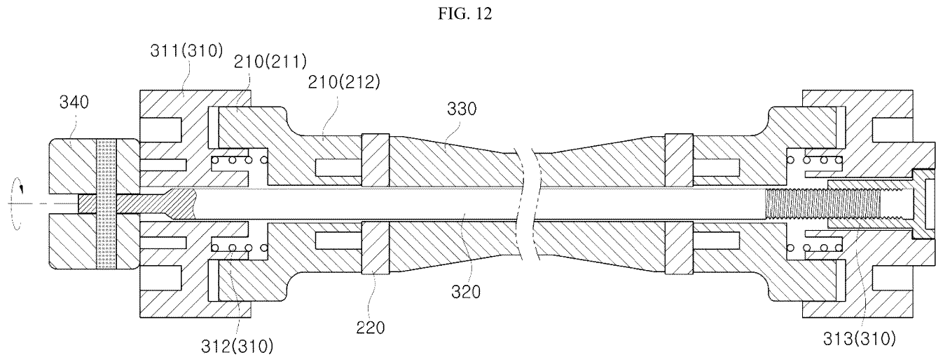

As shown in FIG. 6, the first end and the second end of the base frame 310 include link coupling rings 311 shaped into ring shapes of which longitudinal sections are circular, the link coupling rings 311 having outer side surfaces of a closed structure. Inner surfaces of the outer side surfaces of the link coupling rings 311 may have first gear teeth formed in a circumferential direction thereof. Furthermore, as shown in FIG. 7, the first ends of the first joint links 210 may include base coupling rings 211 which have a ring shape with a circular longitudinal section and smaller than a diameter of each of the link coupling rings 311. Second gear teeth corresponding to the first gear teeth of the link coupling rings 311 may be formed on an outer surface of each of the base coupling rings 211 in the circumferential direction. The base coupling rings 211 may be stored inside the link coupling rings 311. As shown in FIG. 11, the first ends of the first joint links 210 move toward the adjacent first or second end of the base frame 310 (in an outward direction), then when the second gear teeth are engaged with the first gear teeth, the first joint links 210 may be fixed. Meanwhile, as shown in FIG. 12, when the first ends of the first joint links 210 move inward and engagement between the second gear teeth and the first gear teeth is removed, the first joint links 210 may be rotated around the rotation shaft 320.

As an example, as shown in FIG. 5, the second joint links 220 may have a bent bar shape in which the first end portion has a predetermined length and an angle between the first portion and the remaining portion is an obtuse angle. The rotation shaft 320 constituting the base member 300 may penetrate the first end portions of the second joint links 220, and the second ends thereof may be rotatably coupled to a lower surface of a middle portion of the face support fixing plate 400 (i.e., middle portions of the link coupling protrusions 410).

Therefore, the user can adjust angles of the first joint link 210 and the second joint links 220 against the floor surface, thereby adjusting the height and angle of the face support fixing plate 400 fixed, at the upper surface thereof, to the face support cushion 100.

Furthermore, the second joint links 220 may be configured to change an angle of the face support fixing plate 400 by adjusting only the second joint links 220, while a position of the first joint links 210 is fixed with the second end rotated to form an acute angle from the floor surface. To this end, the second joint links 220 shaped into bent bars with the first end portions having a predetermined length and being bent at an obtuse angle from the second end portions may have moving guide holes 221 formed on the first end portions in the longitudinal direction, as shown in FIG. 5. Accordingly, shaft-coupling points between the second joint links 220 and the base member 300 may be moved along the moving guide holes 221. In other words, the rotation shaft 320 may be provided to penetrate the moving guide holes 221, and depending on where the rotation shaft 320 penetrates the moving guide holes 221 by adjusting the second joint links 220, the angle of the face support fixing plate 400 may be changed while the height of the front end of the face support fixing plate 400 is fixed.

Furthermore, in order to stably move the shaft-coupling points of the first end portions of the second joint links 220, the first end portions of the second joint links 220 may be supported such that first side portions thereof are supported by the first ends of the first joint links 210 and second side portions thereof are supported by a bushing 330. For example, as shown in FIG. 7, the first ends of the first joint links 210 may include the base coupling rings 211 protruding outward, and interval adjusting rings 212 protruding inward to be brought into contact with the first end portions of the second joint links 220. Furthermore, as shown in FIG. 11, the base member 300 may include the bushing 330 having a cylindrical container shape having a predetermined length, and provided between the second joint links 220, which face each other, while the rotation shaft 320 penetrates the bushing 330 in a longitudinal direction. Therefore, the first ends of the second joint links 220 may be moved between the interval adjusting rings 212 and the bushing 330 when the shaft-coupling points are moved.

Meanwhile, the base member 300 may be configured to allow respective second ends of the first joint links 210 and the second joint links 220 to be rotated, or maintain the rotated state thereof. To this end, as shown in FIGS. 1 to 4, the base member 300 may include a lever 340 provided on the first end of the rotation shaft 320 protruding outward on the first end of the base frame 310, and the lever 340 may be operated to allow the first joint links 210, the second joint links 220, and the bushing 330 to come into close contact with each other or to release them from the close contact state to shaft-rotate the first joint links 210 and the second joint links 220.

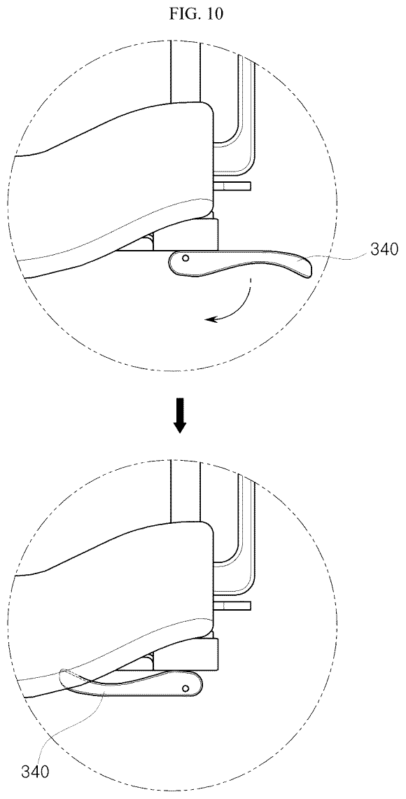

Specifically, as shown in FIGS. 11 and 12, a first end of the lever 340 may be shaft-coupled to a portion of the rotation shaft 320 protruding outward from the first end of the base frame 310, and as shown in FIG. 10, the second end thereof may be laterally turned to face the front side or the rear side. A point of the lever 340 where the lever is shaft-coupled to the rotation shaft 320 may be biased from a center portion of a transverse width of the lever 340. Furthermore, the opposite ends of the base frame 310, the first joint links 210, the second joint links 220, and the bushing 330 may be provided to have gaps therebetween, and elastic members 312 may be provided at the inner portion of the link coupling rings 311 formed at the opposite ends of the base frame 310, as shown in FIGS. 11 and 12, and the elastic members 312 are provided on the inner portion of the first gear teeth to push the base coupling rings 211, which are the first ends of the first joint links 210, by elasticity in a direction where the bushing 330 is provided.

For example, when the lever 340 is laterally turned so that the second end thereof faces the front side, a shaft-coupling point of the lever 340 and the rotation shaft 320 is moved outward more than when the second end of the lever 340 faces the rear side as shown in FIG. 11, so that the rotation shaft 320 is moved in a direction where the lever 340 is provided. A second end of the rotation shaft 320 is coupled to the second end of the base frame 310, and as the elastic members 312 are compressed, the opposite ends of the base frame 310, the first joint links 210, the second joint links 220, and the bushing 330 are brought into contact with each other without gaps. At the same time, the first gear teeth of the link coupling rings 311 and the second gear teeth of the base coupling rings 211 are engaged, thereby maintaining the first joint links 210 and the second joint links 220 in a firmly fixed state without rotation.

Otherwise, when the lever 340 is laterally turned so that the second end thereof faces rearward, a shaft-coupling point between the lever 340 and the rotation shaft 320 is moved inward more than when the second end of the lever 340 faces the front side as shown in FIG. 12, so that the rotation shaft 320 is moved in the opposite direction where the lever 340 is provided. At this point, the elastic members 312 are stretched to generate gaps between the opposite ends of the base frame 310 and the first joint links 210, and at the same time, the first gear teeth of the link coupling rings 311 and the second gear teeth of the base coupling rings 211 are released from engagement, so that the first joint links 210 and the second joint links 220 are brought to be rotated.

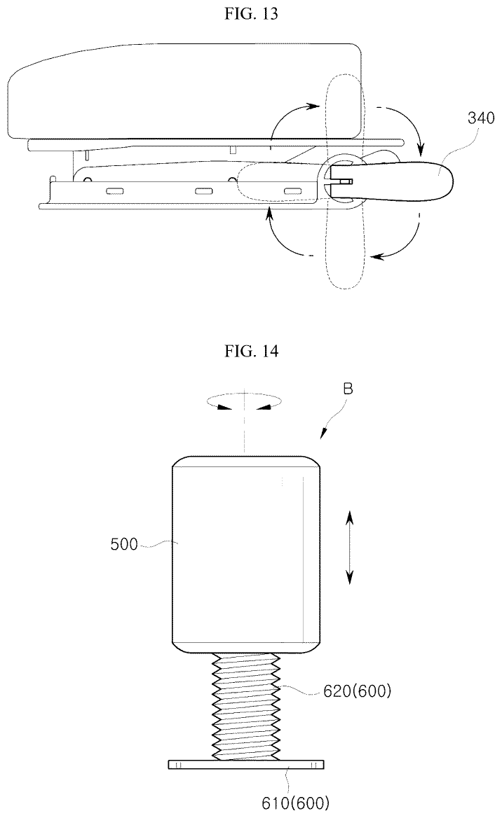

Furthermore, while a shaft-coupling point between the lever 340 and the rotation shaft 320 is moved inward, as shown in FIG. 13, the lever 340 may be rotated around a longitudinal direction of the rotation shaft 320 along with the rotation shaft 320. At this point, as shown in FIG. 12, the second end of the rotation shaft 320 may be screwed to a shaft fixing pin 313 which is fixed not to be rotated to the second end of the base frame 310. Therefore, when the lever 340 is rotated around the longitudinal direction of the rotation shaft 320, a screw-coupling length between the second end of the rotation shaft 320 and the shaft fixing pin 313 is changed depending on a direction where the lever is rotated. Therefore, a degree of acting elasticity by the elastic members 312 may be changed, thereby changing a degree of a force that the user must apply to adjust the first joint links 210 and the second joint links 220.

As described above, the face support module constituting the device for correcting posture according to the present disclosure, as shown in FIG. 9, is configured to be inclined by the link member 200 so that the face support cushion 100 is inclined downward in the forward direction. Accordingly, the user, who lies prone with the head located higher than the feet and the face supported by the face support module A, can be maintained in a posture in which the jaws are pulled and the rear muscles of the neck are relaxed and the ears and shoulders are located at the same level, and while this posture is maintained, stretching using gravity is performed, so that imbalances in the user's body such as turtle neck syndrome and round shoulder can be reduced without stretching that harms the body.

Meanwhile, the device for correcting posture according to the present disclosure may include, as shown in FIG. 9, a shoulder support module B that supports the shoulders of the user lying prone so that the both shoulders of the user lying prone are straightened to use the face support module A.

As an example, as shown in FIG. 14, the shoulder support module B may include a shoulder support cushion 500 on which the shoulder is placed, and a height adjusting stand 600 placed on the floor surface and adjusting the height of the shoulder support cushion 500. Specifically, the shoulder support cushion 500 may have a canister shape, and the height adjusting stand 600 may include a base plate 610 placed on the floor surface, and a height adjusting rod 620 having a rod shape standing upward on a center portion of the base plate 610 and screwed to the shoulder support cushion 500.

Therefore, in order to make the shoulders of the user, who lies prone with the head located higher than the feet and the face supported by the face support module A, the effects of correcting turtle neck syndrome and round shoulder can be maximized by adjusting the height of the shoulder support cushion 500.

1. A device for correcting posture, the device comprising:
a face support module (A) configured to support the face of a user lying prone so that the user's head is located higher than the user's feet,
wherein the face support module (A) comprises a face support cushion (100) having a cross section shaped into a horseshoe configured to support an edge of the user's facial area and a jaw of the user, and a link member (200) configured to tilt the face support cushion (100) so that the face support cushion (100) is inclined downward in a forward direction where the top of the user's head faces, so that the user when lying prone is maintained in a posture in which the jaw is pulled and the ears and shoulders are located on a same level.

2. The device of claim 1, wherein the face support module (A) comprises a base member (300) configured to be placed on a floor, and a face support fixing plate (400) provided above the base member (300) and fixed to the face support cushion (100) at an upper surface thereof,
wherein the link member (200) comprises first joint links (210) and second joint links (220) each having first ends respectively shaft-coupled to portions of the base member (300) located below a rear end of the face support fixing plate (400),
wherein the first joint links (210) are formed longer than the second joint links (220) and second ends of the first joint links (210) are rotatably coupled to a lower surface of a front end of the face support fixing plate (400), and
wherein second ends of the second joint links (220) are rotatably coupled to a lower surface of a middle portion of the face support fixing plate (400).

3. The device of claim 2, wherein the first joint links (210) each has a form of a straight bar, the second joint links (220) each has a form of a bent bar in which a first end portion has a predetermined length and an angle between the first end portion and a remaining portion is an obtuse angle, wherein the first end portions of the second joint links (220) have moving guide holes (221) each formed in a longitudinal direction thereof, and the shaft-coupling points between the second joint links (220) and the base member (300) are movable along the moving guide holes (221).

4. The device of claim 3, wherein the base member (300) comprises a base frame (310) having a ‘ [symbol] ’ shape, a rotation shaft (320) connecting opposite ends of the base frame (310) and penetrating through the first ends of the first joint links (210), which are provided inside close to the opposite ends of the base frame (310), and through the moving guide holes (221) of the second joint links (220) provided inside the first joint links (210), a bushing (330) provided between the second joint links (220) that face each other while the rotation shaft (320) penetrates in a longitudinal direction thereof, and a lever (340) provided at one end of the rotation shaft (320) protruding outward from one end of the base frame (310), which allows the first joint links (210), the second joint links (220), and the bushing (330) to come into close contact with each other or release them from the close contact state to shaft-rotate the first joint links (210) and the second joint links (220).

5. The device of claim 1, further comprising:
a shoulder support module (B) configured to support each shoulder of the user when the user is lying prone,
wherein the shoulder support module (B) comprises a shoulder support cushion (500) configured to support each shoulder of the user, and a height adjusting stand (600) configured to adjust a height of the shoulder support cushion (500).
